(19) United States
(12) Patent Application Publication
(10) Pub. No.: US 2007/0088265 A1
(43) Pub. Date: Apr. 19, 2007

(54) Adapter and pump interface for measuring pressure on an infusion pump

(76) Inventor: Markus Butikofer, Munsingen (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/469,532

(22) Filed: Sep. 1, 2006

Related U.S. Application Data
(63) Continuation of application No. PCT/EP04/14732, filed on Dec. 27, 2004.

(30) Foreign Application Priority Data
Mar. 5, 2004 (DE) 10 2004 010 843.9

(51) Int. Cl. A61M 37/00

(52) U.S. Cl. 604/131

(57) ABSTRACT

An adapter and a pump interface for measuring a pressure associated with an infusion pump including a fluid channel by means of which adapter and pump interface occlusions and/or leaks are rapidly detectable, wherein the adapter includes an insert including a surface which contacts the fluid channel and is movable from a resting position, wherein the pump interface is connectable to the insert of the adapter and includes a sensor, and wherein a fluid pressure can be measured by the sensor in such a way that the pressure is approximately proportional to a force applied to the sensor.

### CROSS-REFERENCE TO RELATED APPLICATION(S)

This application is a continuation of International Application No. PCT/EP2004/014732, filed on Dec. 27, 2004, which claims priority to German Application No. 10 2004 010 843.9, filed on Mar. 5, 2004, the contents of both are incorporated in their entirety by reference herein.

### BACKGROUND

The present invention relates devices for delivering, injecting, discharging, administering, infusing or dispensing substances, and to methods of making and using such devices. More particularly, it relates to infusion pumps and to an adapter and a pump interface for measuring pressure on an infusion pump. The invention relates more particularly to an adapter and a pump interface for measuring a pressure associated with an infusion pump, which are used for detecting occlusions and/or for detecting leaks.

In infusion or injection of a medical or pharmaceutical fluid product or substance, great importance is attached to the exact dosing of a dose or quantity to be administered, particularly in dosed administration to humans. If the administration is performed in a largely automated manner, for example as in insulin treatment using portable infusion pumps, it is then desirable, if not imperative, to monitor the correct administration of the product. One possible monitoring approach is to monitor the pressure of the fluid product since, when the mechanical and electronic components of an infusion or injection appliance are functioning correctly, it is possible to conclude that the product is being administered correctly as long as the fluid pressure remains within a predetermined pressure range. If it does not remain within this range, it is possible to conclude that there is an occlusion or leak present in the fluid conduit system. Particularly in the automated administration of active substance solutions in the medical or veterinary sector, leaks and occlusions pose a considerable risk because of the danger of their not being detected or of their being detected too late.

Designs of infusion pumps and methods for pressure measurement and for occlusion and/or leak detection are known from the prior art.

U.S. Pat. No. 4,373,525 discloses a pressure measurement method which involves detecting the change in the external diameter of an elastic tube through which a fluid intended for infusion passes. The change in the external diameter is correlated with the change in the fluid pressure in the elastic tube.

The method known from U.S. Pat. No. 4,373,525 has been developed in DE 38 71 721 T2 such that, when a predefined threshold value is exceeded, the infusion pump is stopped within a defined time interval and an alarm signal is triggered.

Laid-open specification DE 39 18 534 A1 discloses a pressure sensor for infusion lines which is used for determining the pressure of a medium in an elastic tube section. A pressure member acts on a unilaterally fixed bar carrying an influencing member at its free end. This influencing member is a core of a coil arrangement, the position of the core changing the frequencies of the oscillator coils. Numerous pressure values are recorded at regular intervals and, subsequently, a representative pressure value is obtained by averaging.

European patent 0 291 727 B1 discloses a pressure sensor assembly which is assigned to a driver mechanism for driving a disposable pump cassette comprising a pressure sensor and a position detector, the pressure sensor having an elongate rod-shaped part which is movable in the longitudinal direction and has an end designed such that it can touch the pressure detector of the cassette, the rod-shaped part being designed such that it can move as a function of a change in pressure in the cassette. The assembly further comprises a flexible support for supporting the elongate rod-shaped part, which support can bend as a function of the movement of the rod, and a position detector which can be assigned to the flexible support for monitoring the bending movement of the support. The flexible support has a first flexible support, which is mounted fixedly in the transverse direction of the rod-shaped part, and a second flexible support, which supports a second end of the elongate rod-shaped part, the first flexible support being arranged between the first end and the second end of the rod-shaped part. The first and second supports bend as a function of the movement of the rod-shaped part, caused by a difference in the pressure in the cassette. The position detector is assigned to one of the first and second supports, and an adjustable holding device is provided for calibration as a function of the pressure.

Laid-open specification DE 198 40 992 A1 discloses a process for monitoring the pressure of a fluid product to be administered in dosed amounts during an infusion or injection, which can be dispensed from a container that is received in or formed by a housing, by advancement of a piston received in the container. A reaction force exerted by the piston on the housing serves as a measure for the pressure and is fed to a control for a drive of the piston. The control compares the measured reaction force with a predetermined reference force and controls the drive of the piston taking into account the result of the comparison. The reference force is a nominal value for the reaction force and a direct nominal/actual comparison is carried out between the measured reaction force and its nominal value. The invention further relates to an apparatus which is especially suited for carrying out this process.

U.S. Pat. No. 4,277,227 discloses an infusion pump with adapter. The fluid to be administered flows through a chamber that is widened toward the top. In the event of an occlusion, the level in this chamber rises, and the fluid then makes contact with an elastic membrane. This elastic membrane is expanded and moves a movement device which, for example, is formed by a lever arm. This arm causes a rotating movement of a further element. With the movement of this further element, the position of a light source relative to a photocell is corrected. If light from the light source now falls on the photocell, this optical detection indicates that a pressure threshold value has been exceeded, and an alarm is triggered.

### SUMMARY

An object of the present invention is to measure a pressure associated with a device for delivering a substance, e.g., an infusion pump. Another object of the present invention is to make available an adapter and a pump interface for measuring pressure associated with and/or generated by an infusion pump, whereby a pressure measurement and occlusion detection and/or leak detection can be carried out rapidly and extremely reliably. In addition, the degree of complexity associated with assessing the pressure is greatly reduced, thus resulting in more favorable production costs.

An adapter and a pump interface for measuring a pressure associated with an infusion pump including a fluid channel by means of which adapter and pump interface occlusions and/or leaks are rapidly detectable, wherein the adapter includes an insert including a surface which contacts the fluid channel and is movable from a resting position, wherein the pump interface is connectable to the insert of the adapter and includes a sensor, and wherein a fluid pressure can be measured by the sensor in such a way that the pressure is approximately proportional to a force applied to the sensor.

In one embodiment, the present invention comprises a joining element operably coupled generally between a fluid channel associated with an infusion pump and a sensor.

In one embodiment, the present invention comprises a joining element operably coupled to a fluid channel associated with an infusion pump and a sensor. The fluid channel is connectable to an ampoule and an infusion set and an adapter comprising an insert including a surface contacts the fluid channel and is movable from a resting position. A pump interface is connectable to the insert and comprises the sensor, whereby a fluid pressure associated with the fluid channel can be measured by the sensor in such a way that the pressure is approximately proportional to a force applied to the sensor.

In one embodiment, an adapter according to the present invention for measuring pressure on an infusion pump can, for example, be fitted onto the infusion pump and comprises a fluid channel and an insert piece. A liquid active substance solution is to be moved by the pump, at least a portion of the flow path of the fluid being defined by the fluid channel. The fluid channel has a place where it is connectable to an ampoule, and a place where it is connectable to an infusion set. The ampoule is a reservoir for the fluid, and the infusion set permits infusion of the fluid to a recipient, for example a patient. When the infusion pump and infusion set are functioning correctly, a fluid flows out of the ampoule and through the fluid channel to the infusion set, the fluid being pumped from the ampoule to the infusion set or being displaced out of the ampoule. This pumping process can take place continuously or at intervals. In one embodiment, the insert piece of the adapter according to the present invention has a surface which is in contact with the fluid channel and can, for example, form a boundary of the fluid channel. The insert piece can be made of a single suitable material or of several different materials or composites. If the insert piece surface in contact with the fluid channel is formed by a certain part of the insert piece that can be materially or functionally characterized as a coating, this is interpreted as meaning that the insert piece is in contact with the fluid channel in accordance with the present invention. The insert piece is completely or at least partially movable and can be displaced from a rest position. The rest position comprises the position in which the insert piece is located when it is not subjected to the action of any force from the pressure of a fluid. The fluid pressure in the fluid channel is in this case transmitted directly to the insert piece via its surface, and its surface in contact with the fluid channel in this case serves as a working surface. The insert piece can be composed of one or more individual parts, which can be of a rigid or elastic design.

In some embodiments, the fluid channel of an adapter according to the present invention has a rigid wall. This rigid wall is of advantage, because, in the event of a pressure increase, the fluid pressure can be better transmitted to the insert piece or its working surface. If elastic walls are used, there is a danger that, in the event of a pressure increase in the fluid, these walls could expand excessively, and this could mean the fluid pressure increasing less strongly or with a time delay, even though an occlusion or partial blockage was already present. This can prevent rapid and reliable detection of an occlusion.

In one embodiment according to the present invention, the fluid channel comprises a chamber. In terms of its dimensions, this chamber can differ from the rest of the fluid channel, for example its cross section can be shorter or less than that of the rest of the fluid channel. In some embodiments, it is advantageous if the volume of the chamber is kept relatively small. With a fluid path that is as short as possible, and with a fluid channel whose overall volume is low, an occlusion and/or leak can be detected rapidly and reliably. Accordingly, the chamber should not have too great a dead volume to ensure that the filling volume of the entire fluid channel is not unnecessarily increased beyond what is required. The smaller the volume, the more quickly it is possible to detect a pressure increase within said volume.

In one preferred embodiment of the adapter according to the present invention, the insert piece is arranged in the chamber. For example, the underside of the chamber can be formed by the insert piece. In this case, the surface of the insert piece can be relatively large and flat to permit the best possible transmission of the fluid pressure via its working surface.

In a preferred embodiment of the adapter according to the present invention, the insert piece comprises a ram and/or an adapter membrane. In some embodiments, the ram may be made of a rigid material, and the adapter membrane is elastic and has a low inherent stiffness. The surface of the insert piece in contact with the fluid channel may be formed by the ram. The adapter membrane can be in contact with the fluid channel. In one advantageous embodiment of the present invention, it serves for the elastic support of the ram. The elastically supported ram of the insert piece can be moved out of its rest position by an increased pressure of the fluid. The adapter membrane can, in some partial areas, be fixedly connected to the adapter, so that these areas are not moved. These partial areas may also perform a sealing function.

In one preferred embodiment, the ram is designed as a T-shaped ram comprising an elongate, for example cylindrical part, and a broader top part. The T-shaped ram has an axis symmetry or a point symmetry. The reflection center lies on the longitudinal axis of the ram. The ram can in this case have a rotation symmetry, and it can similarly have a square or rectangular cross section. The ram should be designed such that the fluid pressure transmitted to the surface of the ram does not cause the T-shaped ram to tilt or become wedged, and instead the ram is able to move out of its rest position along a straight axis. In some preferred embodiments, this axis points in the direction of a pump interface according to the present invention.

In one embodiment, the adapter membrane has an approximately U-shaped cross section and is designed extending in a ring shape around the ram. With a suitably angled design of the adapter membrane, for example the U-shaped cross section, good force transmission to the ram is possible. The force acting on the ram is here directly proportional to the prevailing fluid pressure, for example. Upon movement of the ram, the adapter membrane is bent in a predefined manner. The adapter membrane is only insignificantly deformed in the process, i.e. the pressure energy of the fluid is converted only to a small extent into deformation work. Instead, it is practically entirely available for the movement of the ram, thereby permitting a more exact determination of the fluid pressure. In some embodiments, the adapter membrane undergoes practically no expansion.

In a T-shaped ram, the adapter membrane can lie along the elongate part of the T-shaped ram and/or at the same time underneath the T-piece. It is therefore designed extending in a ring shape around a part of the ram that does not have the maximum diameter of the ram, as a result of which, for example, a sealing effect can be achieved.

According to a further aspect of the present invention, it relates to a pump interface with a sensor that can be coupled to the insert piece of an adapter. The coupling can, for example, involve a mechanical contact. The sensor is used to determine the fluid pressure of a fluid whose pressure and force has been transmitted to the insert piece of an adapter. There are different possibilities of directly recording the fluid pressure with a sensor, for example by using pressure sensors or force sensors. Pressure sensors can, for example, be pressure-sensitive microchips with integrated bridge circuit. Their small size is advantageous. Pressure sensors can, for example, be provided with a gel in the housing to protect the sensitive microchip and ensure a uniform distribution of force. They are therefore suitable, for example, for measuring gas and liquid pressures. Some sensors are also mounted resiliently in the housing using a soft adhesive. When acted upon by a force, they are able to deflect to a small extent, or change their position, which is advantageous in the case of mechanical contact by a solid body. A stiff, immovable sensor surface is often not provided. Care should also be taken to contact the microchip parallel or plane on a small surface, for example of 1 mm².

In expansion force sensors, a strain gauge is deformed on a bending beam. Such a strain gauge is, for example, installed in a Wheatstone bridge. A change in tension can be detected that is proportional to the acting force. Because the bending beam, at maximum force, is pressed against a base plate or a limit stop, there is no danger or little danger of destruction as a result of overloading. Depending on the coupling form or depending on the material and physical state of a possible coupling piece, it is recommended to choose a corresponding sensor for realizing the pump interface according to the present invention. It is also possible to connect the insert piece of an adapter directly to the sensor.

According to a preferred embodiment of the present invention, the pump interface comprises a transmission piece via which the insert piece of the adapter can be coupled to the sensor mechanically or magnetically, for example. The transmission piece can have a solid or non-solid physical state. It can be made in one part or composed of several parts, and it can be rigid and/or elastic. The transmission piece can be moved completely or partially out of a rest position. In the rest position, practically no external forces act on the transmission piece. The transmission piece can be coupled to the insert piece, for example in the solid physical state, via a surface which ensures the best possible transmission of force from the insert piece of the adapter to the transmission piece. The setting should be more exact with a punctiform contact.

In an advantageous embodiment of the pump interface according to the present invention, the transmission piece comprises a counter-ram and/or a pump membrane. In some embodiments, the counter-ram may advantageously made of a suitable rigid material, and the pump membrane is elastic and has a low inherent stiffness.

In principle, the counter-ram of the transmission piece can have the same properties and configurations as the ram of the insert piece of an adapter according to the present invention. In principle, the pump membrane of the transmission piece can have the same properties and features as the adapter membrane of the insert piece of an adapter according to the present invention.

According to a preferred embodiment of the pump interface, the counter-ram comprises a dumbbell-shaped counter-ram. The dumbbell-shaped counter-ram has, for example, an elongate, for example cylindrical part, and a broader top part.

According to a preferred embodiment of the present invention, the counter-ram has a symmetrical configuration. As with the ram of an adapter according to the present invention, the center of symmetry of the counter-ram can lie along a longitudinal axis of the counter-ram. The counter-ram can have rotation symmetry and can have a square or rectangular cross section. The counter-ram is designed such that, when force is transmitted from the insert piece of an adapter to the counter-ram, the latter does not experience any tilting and/or wedging, and instead it can be moved without any problem along a defined, for example straight axis.

In some embodiments, the pump interface is designed such that the pump membrane ensures the elastic support of the counter-ram. Analogously to the adapter membrane, the pump membrane too can be clamped securely at an edge area into the pump interface. The clamped part of the pump membrane is thus immovable and cannot be displaced like the counter-ram from a rest position.

In some embodiments, the pump interface according to the present invention is designed such that the pump membrane has a U-shaped cross section and extends in a ring shape around the counter-ram. The U-shape of the cross section or angled configuration of the pump membrane has the effect that, when a force acts on the counter-ram, the pump membrane connected to it is in practice only slightly bent and is not expanded and thus deformed. Therefore, there is practically no deformation work to be performed, and the entire force acting on the counter-ram can be employed to move the counter-ram.

In a preferred embodiment of the pump interface, the pump membrane is designed extending in a ring shape around an area of the counter-ram that does not have the maximum diameter of the counter-ram. In a conventional dumbbell-shaped ram, this means, for example, that the membrane is fitted in an indent in the counter-ram.

The pump interface advantageously has at least one protection element for protecting the pump membrane and/or the sensor from being touched. If touched, damage could be done to the pump membrane, or the sensor could be damaged.

In some embodiments, a protection element for protecting the pump membrane is designed in the form of stirrups, so that touching the pump membrane and/or sensor is ruled out during exchange of the adapter. These stirrups can be arranged parallel to one another and at a certain spacing, for example one that makes contact with a finger impossible. However, they could also be arranged crossing one another. They can be straight or curved.

In one preferred embodiment of the pump interface, the sensor is located on a printed board and is held by a sensor holder. This makes sufficient and exact fixing of the sensor easier, so that an optimum pressure measurement or force measurement can be performed by the sensor. The printed board can, for example, be snapped in a defined manner into the sensor holder. Other suitable securing means, for example adhesive bonding or screws, are also possible.

According to a further aspect of the present invention, the invention relates to a pump with an adapter and with a pump interface as described above. The pump thus at least comprises an exchangeable adapter for measuring pressure, with a fluid channel and with an insert piece, the fluid channel having a place where it is connectable, for example, to an ampoule arranged in a pump, and a place where it is connectable to an infusion set, the insert piece having a surface in contact with the fluid channel and being able to be moved out of a rest position. The pump has at least one pump interface with a sensor which can be coupled to the insert piece of an adapter.

According to the present invention, an ampoule can be inserted into the pump and contains the fluid that is to be administered. To convey the fluid to be administered out of the ampoule, the pump according to the present invention comprises a pump device. This pump device comprises, for example, a plug, a threaded rod and a motor. The plug may be located in the ampoule and it forms, for example, the base of a cylindrical ampoule. The plug can be displaced inside the ampoule by the threaded rod which is driven by the motor. The fluid is forced out of the ampoule by the displacement of the plug inside the ampoule.

In some embodiments, the connection of the fluid channel to the ampoule may be made via a cannula which is formed on the fluid channel of the adapter. This cannula can be guided through a septum that closes the ampoule. This ensures a safe and clean connection.

In some embodiments, the connection of the fluid channel to the infusion set may be made via a Luer connector or Leur-type connector which is enclosed in the adapter. The use of a Luer permits a safe and stable connection of an intrinsically fragile part of the infusion set to the fluid channel of the adapter. Other possible connecting means are also possible, however.

In some embodiments, a pump according to the present invention comprises an adapter membrane and a pump membrane. When the adapter is fitted onto the pump interface, the adapter membrane and the pump membrane are pretensioned slightly relative to one another, so as to more or less compensate for the inherent stiffness of adapter membrane and pump membrane in the zero position. In the zero position, there is an equilibrium of the forces. The compensation of the inherent stiffnesses has the result that, upon a movement of the membrane or of the parts connecting it, for example the insert piece and transmission piece, because of an additional external force or an additional external pressure, such as the fluid pressure, this can be used entirely for displacement of the insert piece and transmission piece. Therefore, no energy is used to overcome the inherent stiffnesses of adapter membrane and/or pump membrane.

In one embodiment of the pump according to the present invention, the inherent stiffness of the pump membrane is less than or greater than that of the adapter membrane. Accordingly, the pretensioning distance of the pump membrane is chosen to be greater or less than that of the pump membrane.

According to a further aspect of the present invention, the invention relates to the use of an adapter for detecting an occlusion and/or a leak. An occlusion signifies a blockage or partial narrowing of the fluid path to an infusion set. The occlusion can be identified through an increase in pressure inside the fluid channel, and can thus be detected by a pressure measurement using an adapter and a pump interface according to the present invention, as described above. An occlusion is present when the measured fluid pressure exceeds a previously specified setpoint value. A leak, that is to say an unwanted loss of fluid product, is present when the measured fluid pressure drops below a previously specified minimum value.

According to a further aspect, the present invention relates to a method for detecting an occlusion and/or a leak using an adapter and/or a pump interface according to the invention as described above. A pressure measurement can be carried out at any desired time, for example by a single measurement. The time at which this single measurement is performed is advantageously independent of the possible intervals at which a fluid medicament is dispensed. The single measurement permits immediate detection of a possible occlusion or leak. The method according to the present invention is thus extremely quick and reliable.

According to a further aspect, the present invention relates to a method for detecting an occlusion and/or a leak using an adapter and a pump interface according to the invention, as described above. In this method, the fluid pressure is proportional, with good approximation in respect of the measurement accuracy of the sensor, to the force that acts on the sensor. This can be achieved, for example, by the fact that, when the adapter is fitted onto the pump interface, the adapter membrane and the pump membrane are pretensioned relative to one another so as to more or less compensate for the inherent stiffnesses of adapter membrane and pump membrane. A corresponding proportionality of the fluid pressure to the force that acts on the sensor can also be achieved by the fact that a membrane which provides an elastic support for a ram has a U-shaped cross section and extends in a ring shape around the ram. In this case, the displaced, rigid ram presses on the sensor. The specific form of the membrane, for example its U-shaped cross section and in general its properties due to its angled structure, ensures that, upon a movement of the ram, the membrane is only slightly bent and is not substantially deformed or expanded. No additional pressure energy is converted into deformation work of the membrane, so that all the energy can be utilized for the displacement of the ram. There is therefore an optimum force transmission, thus ensuring direct proportionality of the fluid pressure to the force that acts on the sensor.

### BRIEF DESCRIPTION OF THE DRAWINGS

FIG. 1 is a cross section through an adapter;

FIG. 2A is a perspective cross section through a pump interface;

FIG. 2B is a perspective view of the pump interface;

FIG. 3A depicts a membrane in a deformed state;

FIG. 3B shows a T-shaped ram supported elastically by a membrane;

FIG. 4 shows characteristic lines of the inherent stiffnesses of membranes;

FIGS. 5A to 5C show membranes pretensioned relative to one another;

FIG. 6 is a diagram of the compensation of inherent stiffnesses of two membranes; and

FIG. 7 shows a pump with adapter and pump interface.

### DETAILED DESCRIPTION

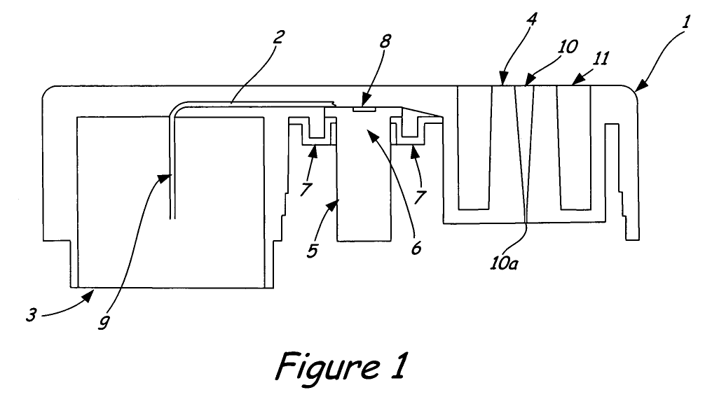

A cross section through an embodiment of an adapter 1 according to the present invention is shown in FIG. 1. It has a fluid channel 2 and an insert piece 5. The fluid channel 2 has a place where it is connectable 3 to an ampoule and a place where it is connectable 4 to an infusion set. The place where it is connectable 3 to an ampoule involves a cannula 9, and the place where it is connectable 4 to an infusion set involves a part 10 of the fluid channel on which a Luer with corresponding Luer thread 11 fits. The fluid channel 2 has a chamber 8, the bottom end of which is adjoined by the insert piece 5. The illustrated course of the flow path or fluid channel can be described as follows:
From the ampoule connection 3 and the cannula 9 into a cylindrical chamber 8. The chamber height is kept very low in order to minimize the filling volume.
Through the chamber 8. The underside of the chamber 8 is delimited by the upper face of the insert piece 5 which, as the working surface, takes over the fluid pressure.
In a channel over the right-hand side of the membrane.
In a perpendicular channel downward along a wall, in order to bypass the Luer thread 11.
In a horizontal channel, past the bottom of the Luer thread 11 (this distance corresponds to the radius of the Luer).
Through a conical or cylindrical hole and upward into the Luer.

The insert piece 5 comprises a T-shaped ram 6 and an adapter membrane 7. The adapter membrane 7 is connected to the T-shaped ram 6 underneath the T-piece of the T-shaped ram. The adapter membrane 7 provides for the elastic support of the T-shaped ram 6. The adapter membrane 7 has a U-shaped cross section and extends sealingly in a ring shape around the T-shaped ram. In this example, the T-shaped ram 6 is rigid, and the adapter membrane 7 has a slight inherent stiffness. Both are made of plastic. The U-shaped cross section of the adapter membrane 7 and its attachment under the T-piece of the T-shaped ram 6 permits a downward movement of the T-shaped ram perpendicular to the fluid channel when pressure is exerted by the fluid, with practically all the pressure energy flowing into the movement work of the T-shaped ram, and with practically no additional deformation work in the adapter membrane 7. When the adapter 1 is used according to the present invention, there is therefore a direct proportional relationship between the fluid pressure in the chamber 8 and the force with which the T-shaped ram 6 is pressed downward.

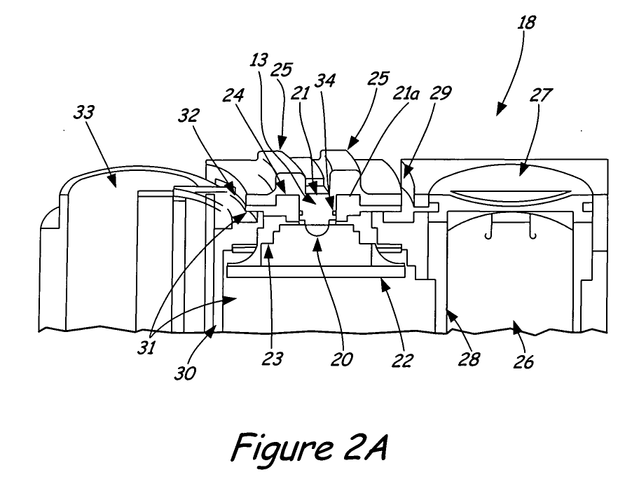

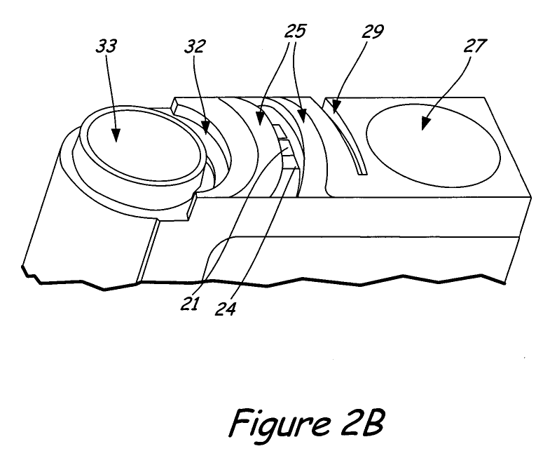

FIG. 2A is a perspective cross section through one embodiment of a pump interface 18 according to the present invention. The pump interface has a sensor 20 that can be coupled to the insert piece 5 of an adapter 1. The sensor 20 is located on a printed board 22, and the printed board 22 is held by a sensor holder 23. In this case, the printed board 22 has been secured on the sensor 23 by being snapped into place. The pump interface 18 has a transmission piece 19. The transmission piece 19 is composed of a counter-ram 21 and of a pump membrane 24. The counter-ram 21 has an indent and is made of a rigid material. The pump membrane 24 is elastic and has a relatively low inherent stiffness. The U-shaped pump membrane 24 extends in a ring shape around the circumference of the counter-ram 21. It engages in the indent in the counter-ram 21. This embodiment of pump membrane 24 and counter-ram 21 ensures that, when force is transmitted to the counter-ram 21 via the insert piece 5 of an adapter 1 according to the present invention, the acting force is used practically exclusively to move the counter-ram 21, and no additional energy is needed for deformation work of the pump membrane 24. A protection element 25 protects the pump membrane 24 and sensor 20 from being touched and/or damaged. The protection element 25 may take the form of stirrups. As regards the periphery of the pump interface 18, further features are disclosed in FIG. 2A. It shows the chassis 31 of the pump interface, and the plastic housing 30. For connecting the pump interface 18 to a corresponding adapter 1, a first adapter connection rail 32 and a second adapter connection rail 29 are in the shape of an arc of a circle. On the left-hand side of the pump interface 18, an ampoule shaft 32 can be seen, into which an ampoule can be screwed. On the right-hand side of the pump interface 18, a battery 26 is located in a battery shaft 28. It is closed off by a battery cover 27.

FIG. 2B is a perspective plan view of the pump interface 18, which has already been shown in cross section in FIG. 2A. This plan view shows, between the two stirrups 25 of the protection element, the top face of the counter-ram 21 around which the pump membrane 24 extends in a ring shape. The first adapter connection rail 32 and the second adapter connection rail 29 can also be clearly seen. The ampoule shaft 32 can be seen on the left, and the battery cover 27 is shown on the right.

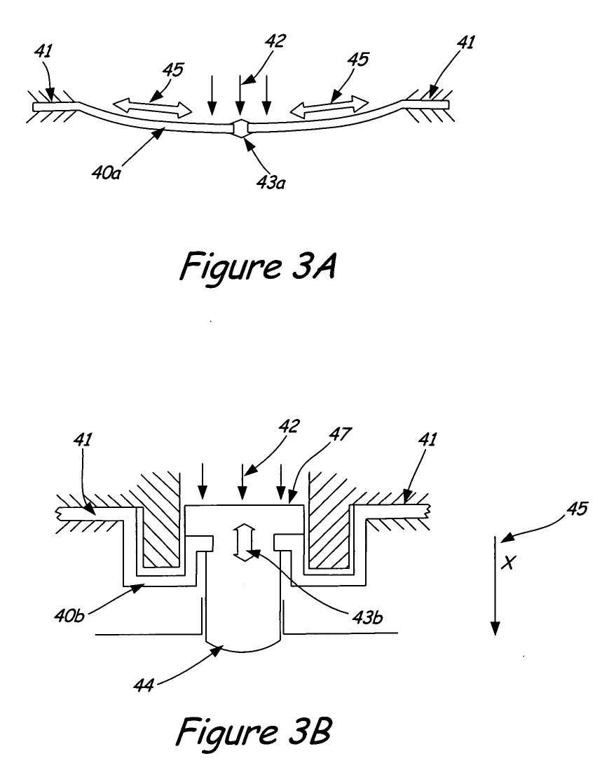

FIG. 3A shows, in cross section, the deformation and expansion of a membrane 40a under the effect of a force 42. The membrane is clamped in place at the sides 41; otherwise it can move freely under the effect of the force 42. The result of the effect of the force 42 can be divided into two components: on the one hand, there is a slight excursion 43a of the membrane 40a in the direction of the acting force 42, and on the other hand the surface of the membrane 40a is expanded, deformation work being required for the expansion 45. The force necessary for the deformation work originates from the acting force 42.

FIG. 3B shows another situation in which a T-shaped ram 44 is supported elastically by a membrane 40b. The situation is shown in cross section. The membrane 40b has a U-shaped cross section. It lies, to both sides of the T-shaped ram 44, underneath the T-piece 47 of the latter and, in three-dimensional view, is designed extending in a ring shape around the T-shaped ram 44. An external force 42 acts on the T-piece 47 of the T-shaped ram 44. The direction of the force 42 is oriented perpendicular to the surface of the T-piece 47 of the T-shaped ram 44. Under the effect of the force 42, the T-shaped ram 44 is moved downward along the X-axis 46. The membrane 40b, with which the T-shaped ram 44 is elastically supported, experiences practically no expansion here; no part of the force 42 is needed to apply energy for a deformation of the membrane 40b. The entire force 42 is available for the downward movement 43b of the T-shaped ram 44. As far as the present invention is concerned, this means that designing the counter-ram or transition piece according to the rules evident from comparison of FIG. 3B with FIG. 3A ensures that the fluid pressure is taken up in an almost optimal manner by a T-shaped ram 44. By providing a construction according to the rules of FIG. 3b, it is thus ensured that the fluid pressure is proportional with good approximation, in respect of the measurement accuracy of a sensor, to the force that acts on the sensor.

Advantageously, in some embodiments both the insert piece 5 of an adapter 1 according to the present invention and the transition piece 19 of a pump interface 18 according to the present invention are equipped with a membrane. The adapter membrane 7 and the pump membrane 24 can have different properties.

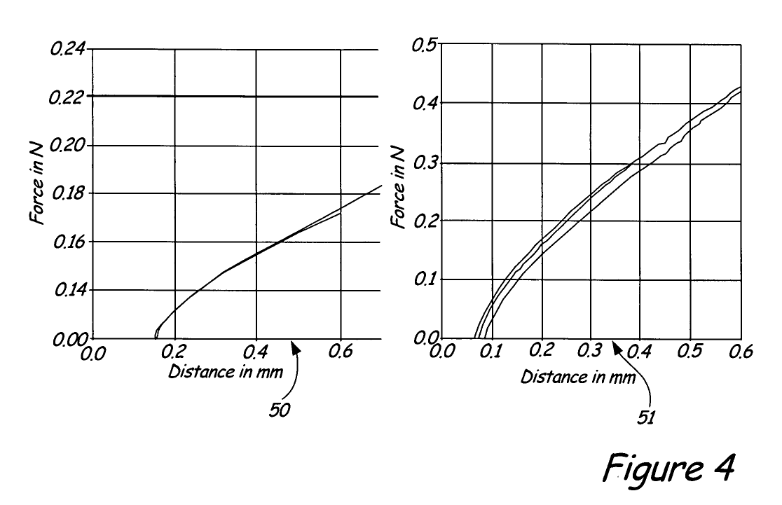

FIG. 4 shows, by way of example, two characteristic lines of the inherent stiffnesses of membranes. The inherent stiffnesses can be determined from the rise of the curves. In both cases, the force needed to obtain a defined movement is plotted against an existing movement. It will be seen that the inherent stiffness of the membrane shown in diagram 50 on the left-hand side is only about half as great as the inherent stiffness of the membrane plotted in the right-hand diagram 51.

Several curves are shown in each of the two diagrams 50 and 51. These curves are each to be assigned to a respective measurement series. They each show a very similar if not absolutely identical characteristic. The inherent stiffnesses of membranes can be reproduced with good accuracy.

When an adapter according to the present invention is fitted onto a pump interface according to the present invention, it may be advantageous to slightly pretension the adapter membrane 7 and the pump membrane 24 relative to one another to obtain a reliable transmission of force to the sensor.

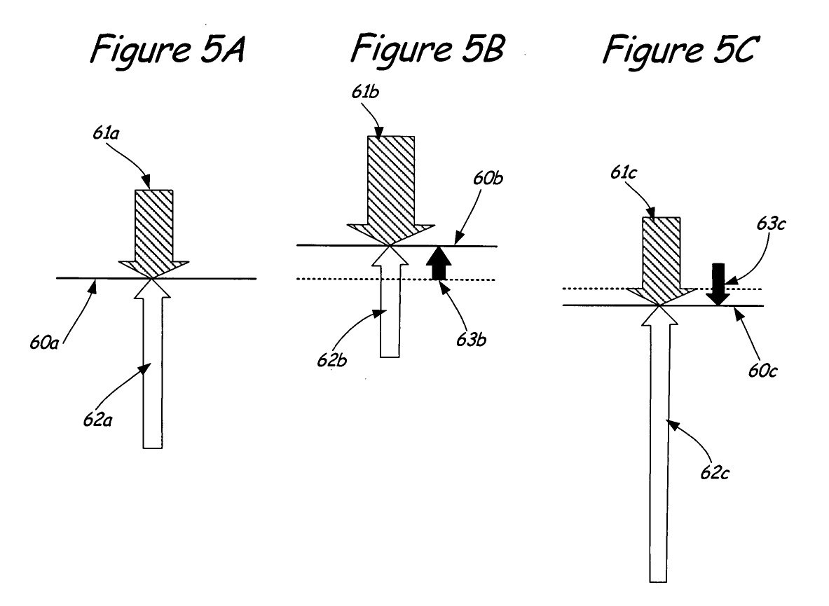

FIGS. 5A-5C show membranes that are pretensioned relative to one another. The forces that occur here are indicated by arrows. The upper, striped arrows 61a, 61b and 61c each designate a membrane force 1, the arrows 62a, 62b and 62c each designate a membrane force 2.

In FIG. 5A, the membrane force 61a and the membrane force 62a are in equilibrium with one another. The system is in the zero position 60a. In this case, no other external forces act on the system.

In FIG. 5B, in addition to the downwardly directed membrane force 61b and the upwardly directed membrane force 62b, there is an external force 63b which is directed upward like the membrane force 62b. The result of this is that the membrane force 61b is increased compared to the membrane force 61a in FIG. 5A, and the membrane force 62b is now smaller than the membrane force 62a in FIG. 5A. The system in FIG. 5B has now moved from the original zero position 60a, and the current zero position 60b has shifted upward. There is an equilibrium of forces in this position 60b.

FIG. 5C shows a movement of the system downward in the other direction. It is in the new zero position 60c under the external action of a force 63c. The membrane force 61c is less than in the case of FIG. 5A, whereas the membrane force 62c is greater than the force 62a in FIG. 5A. The following thus applies in principle: if a membrane is moved in the direction of its pretensioning (that is to say the pretensioning is increased), its force acting on the system increases. A movement counter to the pretensioning of the membrane allows it to relax, and its force becomes less.

In principle, the adapter membrane 7 and the pump membrane 24 are pretensioned relative to one another in such a way that their inherent stiffness is cancelled out at a certain point. If one assumes that the two membranes have different stiffness characteristics, the levels of pretensioning needed for cancelling the force are also different. The point at which the force is cancelled out is called the zero point.

If the pretensioned membrane system is moved upward or downward from the zero point, the different inherent stiffnesses mean that an additional force has to be applied in order to again balance the system that has been shifted out of equilibrium.

If the adapter membrane 7 and the pump membrane 24 are pretensioned relative to one another in such a way that their inherent stiffnesses are more or less compensated, this ensures, for example when the force of a T-shaped ram 6 acts on the membrane system, that only a small amount of force or energy is lost from the system and taken away from the measurement by means of the sensor.

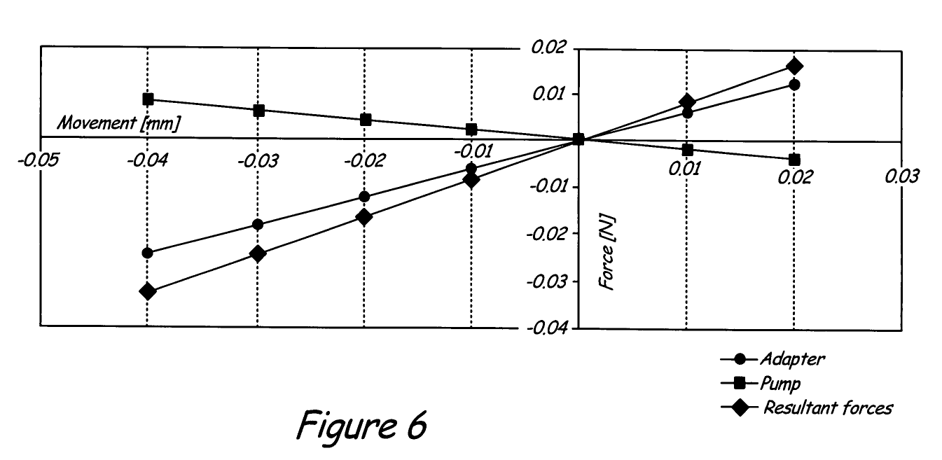

FIG. 6 depicts a diagram for the compensation of inherent stiffnesses of two membranes. In this embodiment, a soft membrane was used for the pump membrane 24, and a, by comparison, harder membrane was used for the adapter membrane 7. The diagram in FIG. 6 shows the resultant forces when the system is moved upward from the zero point and downward from the zero point. The curve belonging to the pump membrane has a comparatively slight negative gradient. The curve of the adapter membrane has a positive gradient which in terms of quantity is considerably greater than the gradient of the curve of the adapter membrane. The curve of the resultant forces is of the greatest interest. The force needed for the movement in accordance with the resultants is the one that is lost from the fluid pressure measurement by the sensor.

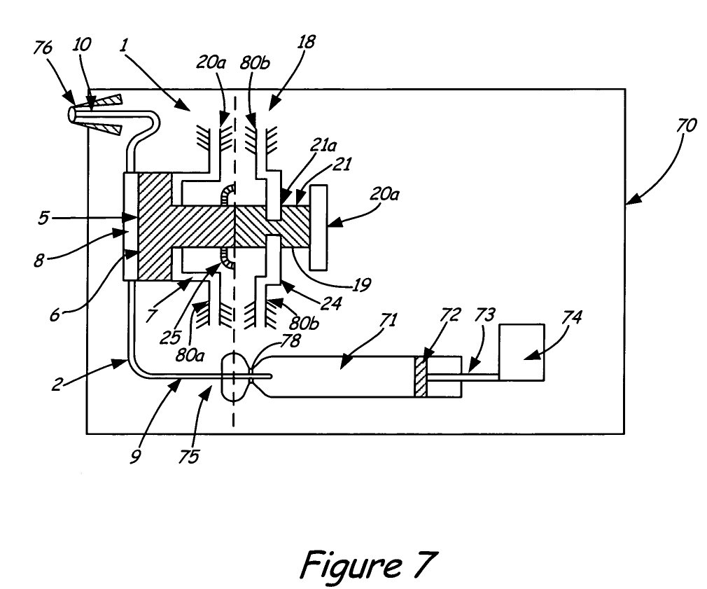

FIG. 7 shows an embodiment of a pump 70 according to the present invention with an adapter 1 and a pump interface 18. The adapter 1 is fitted onto the pump interface 18. An ampoule 71 is inserted into the pump 70. A fluid is forced out of the ampoule 71 by means of a movable plug 72. The plug 72 is coupled to a threaded rod 73 that is moved by a motor 74. The fluid first runs out of the ampoule 71 into the cannula 9, which is attached to the fluid channel 2 of the adapter 1. The cannula 9 pierces a septum 78 with which the ampoule 71 has been sealed. The path of the fluid continues through the fluid channel 2 into a chamber 8. The chamber 8 in this case has a relatively small filling volume, so that a dead volume is kept as low as possible. The path of the fluid then continues through the fluid channel to a connection point 76 for an infusion set, this connection point 76 being forced by a Luer, although other connections are possible. In the chamber 8, the underside of the fluid channel 2 is in contact with an insert piece 5. The insert piece 5 is composed of a T-shaped ram 6 and of an adapter membrane 7. The adapter membrane 7 serves for the elastic support of the T-shaped ram 6. The adapter membrane 7 is clamped securely at the edges 80a into the adapter 1. The ram 6 is made of a rigid material, while the adapter membrane 7 has a low inherent stiffness and a U-shaped cross section. The adapter membrane 7 extends in a ring shape around the T-shaped ram 6 and in addition performs a sealing function. On its underside, the insert piece 5 or T-shaped ram 6 is in contact with a transmission piece 19 of the pump interface 18. The transmission piece 19 comprises a pump membrane 24 and a counter-ram 21. The counter-ram 21 is made of a rigid material and has an indent 21a. In this illustrative embodiment, the counter-ram 21 is dumbbell-shaped. In the indent 21a, the pump membrane 24 is in contact with the counter-ram 21. The pump membrane 24 extends in a ring shape around the counter-ram 21. It serves for the elastic support of the counter-ram and is fixed in the pump interface at the ends 80b. The pump membrane 24 has a low inherent stiffness and a U-shaped cross section. The transmission piece 19 or counter-ram 21 is in contact with a detector 20a for detecting occlusions and/or leaks. To protect the pump membrane 24 and the sensor 20 from being touched, two protective stirrups are mounted as a protection element 25 on the pump interface 18.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An exchangeable adapter for measuring pressure associated with an infusion pump comprising a fluid channel extending between an ampoule and an infusion set, said adapter comprising an insert piece which has a surface in contact with the fluid channel and which can be moved from a rest position.

2. The adapter as claimed in claim 1, wherein at least a portion of the fluid channel has a rigid wall.

3. The adapter as claimed in claim 1, wherein the fluid channel comprises a chamber.

4. The adapter as claimed in claim 3, wherein the insert piece is arranged in the chamber.

5. The adapter as claimed in claim 1, wherein the insert piece comprises a ram and an adapter membrane.

6. The adapter as claimed in claim 5, wherein the adapter membrane elastically supports the ram.

7. The adapter as claimed in claim 6, wherein the adapter membrane has a sealing function.

8. The adapter as claimed in claim 5, wherein the ram is made of a rigid material.

9. The adapter as claimed in claim 5, wherein the ram is T-shaped.

10. The adapter as claimed in claim 5, wherein the ram is axisymmetric or point-symmetric.

11. The adapter as claimed in claim 5, wherein the adapter membrane has a U-shaped cross section and extends in a ring shape around the ram.

12. The adapter as claimed in claim 5, wherein the adapter membrane extends in a ring shape around an area of the ram that does not have the maximum diameter of the ram.

13. A pump interface comprising a sensor which can be coupled to an adapter comprising an insert piece.

14. The pump interface as claimed in claim 13, further comprising a transmission piece via which the insert piece of the adapter can be coupled to the sensor.

15. The pump interface as claimed in claim 14, wherein the transmission piece comprises a counter-ram and a pump membrane.

16. The pump interface as claimed in claim 15, wherein the pump membrane elastically supports the counter-ram.

17. The pump interface as claimed in claim 16, wherein the counter-ram is made of a rigid material.

18. The pump interface as claimed in claim 15, wherein the counter-ram comprises an indent.

19. The pump interface as claimed in claim 18, wherein the counter-ram is symmetrical in respect of a point reflection.

20. The pump interface as claimed in claim 17, wherein the pump membrane has a U-shaped cross section and extends in a ring shape around the counter-ram.

21. The pump interface as claimed in claim 18, wherein the pump membrane extends in a ring shape around the indent of the counter-ram.

22. The pump interface as claimed in claim 21, further comprising at least one protection element associated with the pump interface for preventing the pump membrane from being touched.

23. The pump interface as claimed in claim 22, the protection element comprising stirrups arranged such that touching at least one of the pump membrane and the sensor is prevented when exchanging the adapter.

24. The pump interface as claimed in claim 13, wherein the sensor is located on a printed board and is held by a sensor holder.

25. A pump comprising a fluid channel, an adapter comprising an insert piece which has a surface in contact with the fluid channel and which can be moved from a rest position, and a pump interface comprising a sensor which can be coupled to at least one of the adapter and the insert piece.

26. The pump as claimed in claim 25, wherein the fluid channel comprises a cannula extending through a septum that closes the ampoule.

27. The pump as claimed in claim 26, wherein the fluid channel extends to an infusion set coupled to the cannula by a Luer connector enclosed in the adapter.

28. The pump as claimed in claim 27, wherein the adapter comprises an adapter membrane and pump membrane, said adapter membrane and pump membrane pretensioned slightly against one another when the adapter is fitted onto the pump interface so as to compensate for the inherent stiffnesses of adapter membrane and pump membrane.

29. The pump as claimed in claims 28, wherein the inherent stiffness of the pump membrane is less than that of the adapter membrane.

30. A method for detecting an occlusion or a leak associated with a device for discharging a fluid at intervals, the device comprising a fluid channel, said method comprising the steps of:
providing an adapter comprising an insert piece which has a surface in contact with the fluid channel and which can be moved from a rest position;
providing a pump interface comprising a sensor which can be operably coupled to at least one of the adapter and the insert piece, and
carrying out at least one pressure measurement at any desired time independently of the intervals at which a fluid is discharged.
